(19) United States
(12) Patent Application Publication
(10) Pub. No.: US 2026/0174384 A1
(43) Pub. Date: Jun. 25, 2026

(54) Substance Delivery Device and Methods of Enforcing Therapeutic Adherence

(71) Applicant: Perry S. Kaye, Cooper City, FL (US)

(72) Inventor: Perry S. Kaye, Cooper City, FL (US)

(21) Appl. No.: 19/541,390

(22) Filed: Feb. 16, 2026

Related U.S. Application Data
(63) Continuation-in-part of application No. 17/731,256, filed on Apr. 27, 2022, now Pat. No. 12,551,634.
(60) Provisional application No. 63/180,654, filed on Apr. 27, 2021.

(51) Int. Cl. A61B 5/00; G16H 20/13

(52) U.S. Cl. CPC A61B 5/4833; A61B 5/4839; G16H 20/13

(57) ABSTRACT

A substance delivery device and methods to promote treatment regimen compliance. A beneficial substance and an addictive substance are administered from a device delivering the substances by a user inhaling vaporized or atomized beneficial substances and addictive substances per prescribed treatment regimen protocol that prevents underdosing and overdosing of a therapeutic substance.

### CROSS REFERENCE TO RELATED APPLICATION

This continuation-in-part application claims priority to U.S. provisional application No. 63/180,654 having a filing date of Apr. 27, 2021, titled Substance Delivery Device and Methods, to U.S. Non-provisional application Ser. No. 17/731,256 entitled the same and having a filing date of Apr. 27, 2022, the entire contents of which are incorporated herein by reference.

### FIELD OF THE DISCLOSURE

The present disclosure relates to devices and systems for delivering a substance to a user and methods of use thereof. More specifically, the devices and methods pertain to a system to encourage behavioral traits, routines, and treatment compliance by pairing an addictive or desirable substance with a therapeutic substance in a vaporization or atomization delivery device.

### BACKGROUND OF THE DISCLOSURE

Users often use chemicals, like medical drugs, medicinal drugs, nutritional supplements, or combinations thereof, to achieve positive and beneficial outcomes. Medical treatments, medicinal treatments, nutritional regimens, and combinations thereof are designed to increase the chemical's effectiveness and confer a positive benefit on the user. While chemicals, including drugs like, but not limited to insulin, antiviral, antibacterial, antidepressants, hormones, antipsychotics, cancer treatments, and nutritional supplements like but not limited to vitamins, minerals, and extracts often perform specific tasks it is a well-known fact that they can only work if used properly and in specific manner. The chemicals cannot work if they are not taken. They can sometimes do harm if are taken in too large a quantity or with other chemicals.

To determine if a patient is responding to the treatment, it is common for testing is often also performed to determine if the chemical is working as expected or not.

For the chemicals to be effective they must often be taken over set periods of time, at set intervals and in specific dosages. Failure to comply with dose, timing, and/or intervals, in a coordinated manner can cause the chemicals to be too weak or too strong and this can cause the user to not achieve the intended benefit or worse to injure themselves. Too little of a compound can often cause the compound to be ineffective. And too much of the compound can sometimes be deadly.

Mixing chemicals can either be nonreactive, can negate the effects of one or more of the chemicals, or can modify the effects of one or more of the chemicals in either a positive, negative, or random manner. Mixing chemicals can be intentional to elicit a desired outcome or accidental which can lead to negative results including death and/or injury.

A trained medical professional will often do a drug utilization review (DRU) to ensure a person's drug regimen has the highest chance to ensure the person the best possible clinical outcome and also to avoid interactions.

A DRU of a person's regimen should take into account the best way to administer each chemical and also how the chemicals the patient will take may interact when taken together. For example, it is common for some medications to require them to be taken on a full stomach while others must be taken on an empty stomach. Many types of interactions must be avoided including for example drug to drug interactions which occur when the combination of the chemicals produces an unwanted result, injury, or alters the function of the chemical. Another type of interaction is the drug to disease; where a patient's disease may be made worse by a particular drug or the drug may be too strong for an infant or child. And another is a drug to patient; for example, where a drug may harm a pregnant individual or their unborn baby. Different patient's allergies, gender, pregnancy, age (pediatric or geriatric or etc.), condition, or genetics can alter the chemical's effect on the person and must be considered. A DRU works to make sure the chemicals the person is taking have the least, preferably none, interactions, and have the best chance of success for the person. Therapeutic adherence remains a critical challenge in modern healthcare, with studies indicating that approximately 50% of patients do not follow prescribed medication regimens adequately. This non-compliance results in significant healthcare costs, treatment failures, and adverse patient outcomes. Traditional approaches to improving therapeutic adherence have focused on patient education, reminder systems, and simplified dosing schedules, but these methods have proven insufficient for achieving consistent long-term compliance.

Existing medical devices and systems for medication delivery typically operate without incorporating mechanisms that create sustained user engagement or dependency on the delivery system itself. While some devices attempt to improve adherence through automated dispensing or reminder notifications, they fail to address the fundamental issue of user motivation and behavioral compliance. The lack of intrinsic user dependency on the dosing device results in continued non-adherence when external reminders or incentives are removed.

Current pharmaceutical and medical device approaches also suffer from the limitation that users may discontinue use of the dosing device once they achieve their therapeutic goals, or when they no longer require external reminders. This discontinuation leads to relapse or treatment failure, particularly in chronic conditions or addiction management.

Non-adherence to prescribed medication regimens is a pervasive and critical challenge in modern healthcare. It is estimated that a significant percentage of patients fail to take their medication as prescribed, leading to suboptimal clinical outcomes and increased healthcare costs. The World Health Organization and subsequent peer-reviewed studies have identified non-adherence as a leading cause of hospitalization, treatment failure, and preventable mortality.

The economic burden of non-adherence is substantial. Medication non-adherence is associated with elevated mortality, increased healthcare utilization, and significant direct and indirect financial costs to patients and society. This issue remains under-addressed in clinical settings despite its far-reaching consequences.

Current strategies to address non-adherence primarily rely on behavioral interventions, including patient education, reminder systems, and increased “patient engagement.” While these methods aim to improve awareness, they are fundamentally passive. They require the patient to exercise cognitive effort and willpower to initiate the dosing process.

The efficacy of these traditional methods is limited by the physiological and cognitive state of the patient. Illness often induces cognitive impairment, fatigue, and emotional distress, which diminishes the capacity for self-regulation. As noted in clinical literature, the complexity of modern therapeutic regimens places a heavy cognitive load on the patient, making compliance difficult even for healthy individuals.

Testing how a treatment regimen is working for a patient is dependent on the treatment regimen being followed. For example, one cannot know if a person's health is improving due to taking a particular drug if you are not able to determine if, in fact, the person is taking the drug. In addition to taking the chemicals the patient's body may not be absorbing or reacting as intended to the chemical. Knowledge of the treatment plan and the testing results enable the plan to be modified so that it has its best chance of working and also least chance of doing harm.

A treatment plan must be followed to work. A treatment plan must be managed and adjusted based on testing. Non-adherence is a major problem that limits the effectiveness of many treatment plans. And also causes problems where medical and nutritional professional may make dangerous or erroneous suggestions based on the false data non-adherence generates. For example, a doctor may increase a person's medications based on the assurances that the patient is taking a medication when in fact they are not taking the medication. This can be dangerous for the user and for the public.

Patients often want to comply with treatment regimens but there are many reasons why they are unable and shift into non-adherence. Willpower exhaustion, a lack of planning, disorganization, misunderstanding the dosing instructions, forgetfulness, running out of the drug, and other reasons make coordination and administration of the regimen to be burdensome. It is often especially difficult for people with mental illness diseases to track, plan, dose, and continue to adhere to the treatment plan. For example, people with bipolar, schizophrenia, or schizoaffective disorders may find it especially difficult to remain compliant to a designed regimen when they are feeling okay or feeling normal. It is essential for many people, often life and death, to adhere to their treatment regimes.

Too many people fail at treatment adherence because it is a task that takes time, energy, organization, and must be done consistently. It is a massive effort that is difficult for healthy people to follow and even more difficult for ill people to follow. The failure to comply with a drug regimen result in increased costs from the providers and expenses from the user.

Consequently, there is a critical need in the art for a system that ensures therapeutic compliance without relying on the patient's willpower or voluntary action. Existing solutions fail to address the root cause of non-adherence: the lack of an automated mechanism to compel adherence. There is a need for a novel approach that utilizes physiological dependencies to ensure that patients receive their prescribed medication, thereby optimizing therapeutic efficacy and patient outcomes.

### SUMMARY OF THE DISCLOSURE

The need exists for a system and method that creates a pharmacological dependency relationship between the user and the dosing device itself, thereby ensuring sustained therapeutic adherence through automatic and opportunistic dosing mechanisms. Such a system would leverage the user's dependency on the dosing device to maintain compliance with therapeutic regimens, even in the absence of external motivation or reminder systems. More specifically, what is needed is needed is a substance delivery device having vaporization or atomization capabilities paired with at least one substance cartridge comprising a beneficial substance and an addictive substance. A user places a mouthpiece of the device in his/her mouth to draw air through an air intake comprising a predetermined concentration of a beneficial substance and an addictive substance, or an addictive substance and a placebo, or a beneficial substance and a placebo.

The device comprises external control, connectivity, and notification features to promote compliance and to collect, process, and send regimen compliance information to a health care provider or prescriber.

The predetermined concentrations and dosages for each substance or placebo is regulated by the device's computer system. A prescriber or treatment provider programmatically configures the dose, concentration, and frequency. The instructions are stored in a data storage and processed by a controller and memory.

A beneficial substance is prescribed and filled or otherwise obtained in a cartridge. The cartridge comprises at least two bladders or a bifurcated chamber to contain the beneficial substance and the addictive substance. However, in other embodiments, the beneficial substance and the addictive substance may be mixed and contained in one cartridge bladder. In some embodiments, a cartridge comprises an addictive substance, a beneficial substance, and a placebo.

### BRIEF DESCRIPTION OF THE DRAWINGS

The accompanying drawings that are incorporated in and constitute a part of this specification illustrate several embodiments of the disclosure and serve to explain the principles of the disclosure.

FIG. 1 illustrates an exemplary schematic of a system is shown.

FIG. 2 illustrates a perspective view of an exemplary device with external components.

FIG. 3 illustrates an internal front and side view of an exemplary cartridge.

FIG. 4 illustrates a front interior view of an exemplary device with a cartridge.

FIG. 5 illustrates an exemplary system flow chart.

FIG. 6 illustrates an exemplary method steps schematic.

FIG. 7 illustrates an exemplary method steps schematic with an additional weaning step.

FIG. 8 illustrates alternative exemplary method steps.

FIG. 9 illustrates a schematic including an inactive ingredient and a weaning step.

FIG. 10 illustrates a schematic with a delay timer function.

FIG. 11 illustrates an exemplary system central processing unit.

FIG. 12 illustrates exemplary substance cartridges.

FIG. 13 illustrates an exemplary device.

FIG. 14 illustrates an exemplary device with security fob.

FIG. 15 illustrates exemplary method steps for a provisioning procedure.

### NUMERALS OF THE FIGS

001 atomizer/vaporizer device (“device”) computer
100 user
105 beneficial substance
110 beneficial substance dose release
115 beneficial substance use regimen
120 addictive substance
125 addictive substance dose release
130 addictive substance use regimen
140 software
150 controller
145 data storage
155 memory
200 atomizer/vaporizer device (“device”)
203 device housing
205 control scroll PREVIOUS button
206 control scroll NEXT button
207 control SELECT button
208 display
210 speaker
215 cartridge connector
220 substance in-port
225 light-emitting diode
230 data port
235 optical encoder
240 drive shaft
245 battery
250 mouthpiece out-port
255 cartridge location 1
256 cartridge location 2
257 cartridge location 3 (Not Shown)
258 cartridge location 4 (Not Shown)
260 Bluetooth notification
265 WiFi notification
270 power button
300 cartridge
305 substance bladder
310 substance fill port
315 substance feed line
320 rotor
325 roller
330 shaft hole
335 snap connector
340 cartridge out port
345 rotor alignment spindle
350 cartridge data chip
360 light-emitting diode lens
400 heating element
410 heating chamber
415 substance check valve CLOSED
416 substance check valve OPEN
420 air intake aperture
425 air check valve
430 pressure sensor
440 inhale check valve
450 mouthpiece
455 vapor out-port
500 user
510 substance prescriber
515 substance prescriber prescribes beneficial regimen for user
520 beneficial substance regimen is created
530 pharmacist/dispensary
525 pharmacist/dispensary fill prescription making the cartridge
536 filling cartridge with beneficial substance
537 endowing of beneficial regimen into cartridges data chip
538 optionally printing label containing beneficial regiment data for user or attaching to cartridge
540 user takes custody of cartridge
550 user attaches cartridge to device
555 device validates cartridge using data in the device, in the cartridge data chip and optionally from the cloud or an app
560 determining whether cartridge valid determination is made
565 determining that if the cartridge is not valid then an error condition is created and relevant parties are notified
570 determining if the cartridge is valid then the user is told they are able to use the device and cartridge

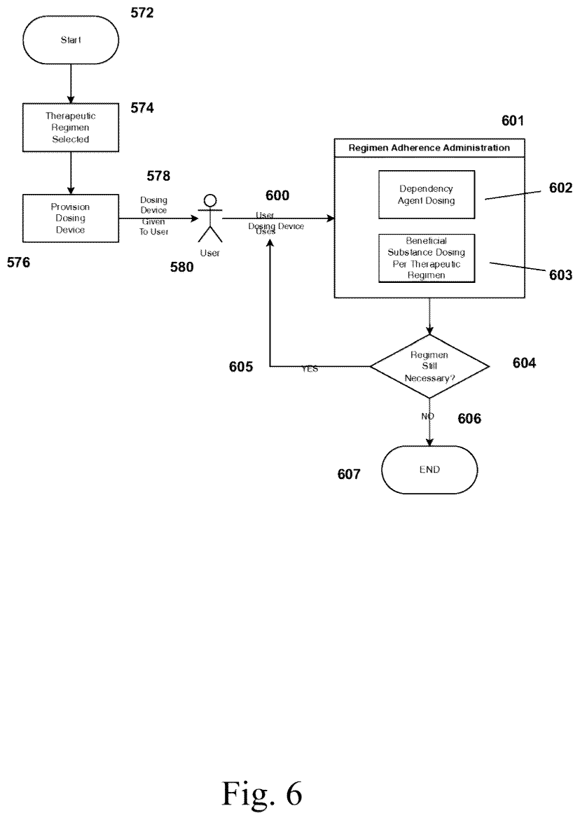

The remaining numerals for FIGS. 6-15 are provided in the text of the detailed description.

### Definitions

“Dependency Agent” and “Addictive Agent” and “Dependency Substance” and “Addictive Substance” as used herein refers to a pharmaceutical agent or compound or combination of compounds that exhibits significant physiological or psychological dependency characteristics, including but not limited to substances classified as stimulants, depressants, opioids, benzodiazepines, cannabinoids, antipsychotics, antidepressants, mood stabilizers, and anticonvulsants. A Dependency Agent may be a single active pharmaceutical ingredient or a combination thereof and includes substances that demonstrate measurable levels of physiological dependency (as defined by severity of withdrawal symptoms, tolerance development, and physical dependence indicators) and/or psychological dependency (as defined by compulsive use patterns, craving behaviors, and behavioral reinforcement mechanisms). The Dependency Agent encompasses both licit pharmaceutical compounds and illicit substances that exhibit the pharmacological characteristics of dependency, including but not limited to nicotine, alcohol, opioids (such as heroin, fentanyl, oxycodone), benzodiazepines (such as diazepam, alprazolam), caffeine, sugar, stimulants (such as amphetamines, methamphetamine), cannabis, selective serotonin reuptake inhibitors (SSRIs), antipsychotics (such as risperidone, olanzapine), mood stabilizers (such as lithium, valproate), and anticonvulsants (such as carbamazepine, valproic acid). The Dependency Agent may be administered alone or in combination with other agents to achieve desired pharmacological effects while accounting for its inherent dependency potential and associated withdrawal characteristics.

“Beneficial Substance” and “Beneficial Agent” and “Therapeutic Substance” and “Therapeutic Agent” as used herein refers to a compound or combination of compounds that provides therapeutic, medicinal, nutritional, or beneficial effects to a patient or subject. A Beneficial Substance may be a naturally occurring compound, synthetic pharmaceutical agent, nutraceutical, dietary supplement, or combination thereof, that demonstrates therapeutic efficacy, nutritional value, or beneficial effects for treating, preventing, or managing specific medical conditions, disorders, or for health maintenance needs.

The Beneficial Substance encompasses active compounds that produce desired therapeutic outcomes, nutritional benefits, or health-promoting effects through mechanisms of action. Such substances include but are not limited to vitamins, minerals, herbal extracts, amino acids, fatty acids, probiotics, prebiotics, antioxidants, botanical compounds, essential oils, water, electrolyte solutions, dietary fibers, and other naturally occurring or synthetic compounds that provide medical, nutritional, or health benefits.

The Beneficial Substance may be administered alone or in combination with other therapeutic agents to achieve desired medical outcomes, nutritional support, or health maintenance. The term encompasses established pharmaceutical compounds, novel therapeutic agents, nutraceuticals, dietary supplements, functional foods, and other non-medical agents that demonstrate substantial therapeutic benefit, nutritional value, or health-promoting properties. This definition specifically includes water as a Beneficial Substance, recognizing its essential role in health maintenance and hydration.

“Time Mechanism” as used herein refers to a timekeeping mechanism that provides temporal awareness and synchronization capabilities for the CPU and software operations to ensure beneficial substance dosing is performed per therapeutic regimen adherence. The time mechanism may be implemented through various technologies including but not limited to crystal oscillators, real-time clocks (RTC), microcontroller-based timing circuits, software routines that request time from a known time source like a web based atomic clock, or software-based timekeeping modules that maintain temporal accuracy for the device's operational timing requirements.

“Inactive Ingredient” as used herein refers to any substance that is not provided for therapeutic benefit nor dependency-inducing properties. Substances like flavorants, aromatics, water, propylene glycol, vegetable glycerin, and other chemicals that can be used to either 1) enhance the dosing experience (as flavorings May 2) to enable a person to take a dose that is does not include a beneficial nor addictive substance.

“Therapeutic Regimen” and “Beneficial Regimen” as used herein refer to a beneficial regimen that includes at least one beneficial substance, a recommended dose for each beneficial substance, and a frequency of use for each beneficial substance. This may be a doctor's prescription, a nutraceutical regimen, or a user selected regimen. One example of a beneficial regimen is a low-dose aspirin regimen: beneficial substance=aspirin, dose=81 mg, frequency=daily. Another example of a beneficial regimen is a hydration regimen: beneficial substance=water, dose=1 cup, frequency=10 times per day.

“Opportunistic Dosing” as used herein refers to automatically, simultaneously, and per the therapeutic regimen dosing the beneficial substance while the user is using the dosing device to satisfy their physiological and pharmacological dependency on the dependency agent.

### DETAILED DESCRIPTION

The present disclosure relates to a system and method for ensuring therapeutic adherence through use of an intentional addiction to the dosing device and automatic opportunistic dosing of beneficial substances per the therapeutic regimen. Specifically, the invention provides a system that leverages an intentional dependency on the dosing device to ensure a patient adheres to a prescribed medication regimen.

The system comprises a medication delivery device configured to administer one or more therapeutic agents according to a prescription, and one or more addictive or reinforcing agents. The device includes a controller configured to manage the dosing of a beneficial substance per a therapeutic regiment while the user is using the dosing device to satisfy their physiological dependency on the device. The system is operable to administer the addictive agent in a dosage sufficient to create a dependency, thereby biasing the user to utilize the device. As the user uses the device the device opportunistically doses them their medication per the prescription.

The dosing device can be any device that can administer at least one addictive dependency agent and at least one beneficial agent per a therapeutic regimen. Dosing devices in the form of a vape, a dosing water bottle, dosing nasal sprayer, an inhaler, a nebulizer, and other devices suitable to the user, therapeutic regimen, beneficial substance, and dependency agent.

A key feature of the invention is a closed-loop feedback and control mechanism. The system is adapted to continuously monitor patient interaction with the device and compare said interaction against the prescribed therapeutic parameters. In response to a determination of non-adherence (e.g., missed or delayed administration of the therapeutic agent), the system is programmed to escalate the administration of the addictive agent to reinforce behavioral compliance. Conversely, upon detection of excessive usage exceeding therapeutic requirements, the system is adapted to modulate the addictive agent to maintain a controlled, reversible dependency while ensuring optimal therapeutic delivery. while keeping the users use optimized to enable opportunistic dosing per the prescription.

The system is further configured to allow for the dynamic selection and adjustment of the addictive agent dosage, independent of the therapeutic agent dosage. This allows the Regimen Prescriber to tailor the treatment to the patient, utilizing variables such as body size, metabolism, prior use of addictive substances, and response to therapy. The addictive agent can be raised, lowered, switched, combined with other agents, or discontinued while maintaining the dependency on the device, ensuring that the patient remains compliant and adherent with the therapeutic regimen.

In some embodiments, addictive substance to create an addiction schedule that is aligned with a subject's desired healthful regimen. Using the compelling power of an addiction to create the act of dosing with a device and then also delivering a beneficial substance at set times or based on other pre-determined parameters can greatly help a user adhere to his/her treatment.

The disclosure relates to using a controlled addiction to bias a person to comply with necessary drug regimens. Patients who rely on drugs to maintain their health but are unable to or find it difficult to maintain proper medical drug use schedules could be helped by a strong and controlled addiction that aligns with their drug use schedule and a drug metering system that dispenses medical drugs along with the controlled addictive substance.

For example, nicotine is a highly addictive substance with mild side effects. Exemplary devices utilize a pre-programed metering system to dispense a prescribed dose of at least one beneficial substance as well as a controlled addictive substance to bias the person to use the system and thus achieve treatment adherence.

In one regimen example, a user is prescribed critical medication to promote mental stability or to improve chemical imbalances in the central nervous system. An exemplary device that has two or more tanks, one including the critical medication or beneficial substance and the other including synthetic or naturally-derived nicotine, pre-determined treatment regimen software instructions that are processed and control the dosage and concentration from the bipolar med tan. The device monitors via sensors and collects information in a tracking system that ensures the user is getting the correct dose of the beneficial substance. In addition, a device may contain a sensing system to check the user's vitals and may contain an notification or alert system.

The user will begin using the device and will get notices (via sounds, light, text, or etc) and will be dosed with nicotine and/or the beneficial substance depending on the dosage needs of the user and the need to maintain the addiction for the user's benefit. As the user uses the device, it will track their use and either dose nicotine and the beneficial substance, nicotine only, or beneficial substance, and as necessary. In some embodiments, a placebo may be added and dispensed as well, especially for weaning and reducing tolerance build ups.

As the user uses the device, a profile is generated of the user and a dosing schedule is formed that determines correct doses of nicotine and beneficial substance to bias the user to their scheduled medical regimen. All information is tracked in the device and can be reviewed and updated by a medical professional.

The device can also notify others if the person is out of compliance. The device can also gamify the use of the device to make it fun to use. Sensors in the mouthpiece and/or grip can test the person's state (i.e. breath and saliva and temperature and heart rate and etc.) and track that information and then adjust doses based on the information.

Additional tanks can be added to enable a person to be given multiple doses of different drugs so their entire drug/beneficial substance regimen, including medical and supplemental (vitamin D, cod liver oil, etc.) could be included in the program. The system would have beneficial substances in tanks that included dosing information, batch numbers, serial numbers and other related information in a form readable by the device dispensing the substances via atomization or via vaporization.

Addictive substances may be nicotine, caffeine, sugar, or any other suitable excipient. Beneficial substances may be any liquid suspension or mixture suitable for oral or nasal administration.

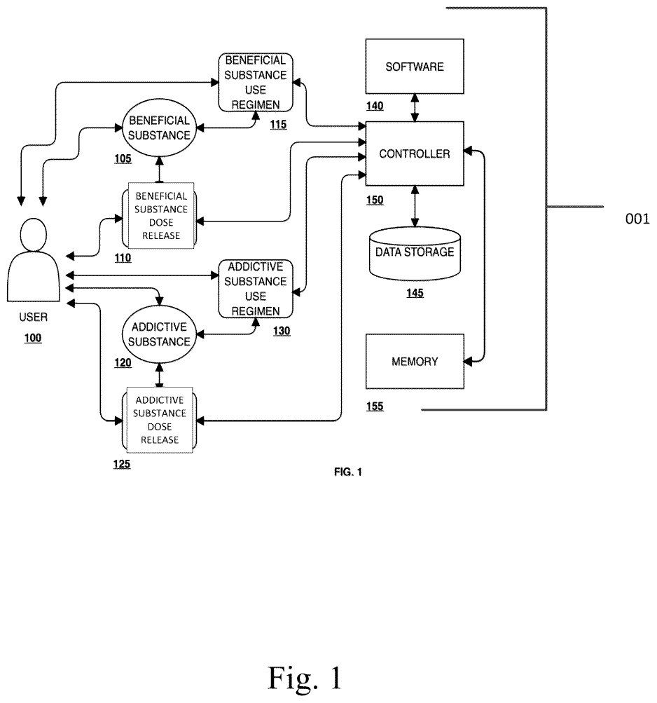

Referring to FIG. 1, an exemplary system schematic is shown. User 100 will take Beneficial Substance 105 in accordance with Beneficial Substance Use Regimen 115 via Beneficial Substance Dosing Means110. To assist User 100 in maintaining compliance with Beneficial Substance Use Regimen 115 this system uses an Addictive Substance 120 according to an Addictive Substance Use Regimen 130 that is administered via an Addictive Substance Dosing Means 125.

Controller 150 reads Beneficial Substance Use Regimen 115 and Addictive Substance Use Regimen 130 to determine how to best meet the User 100 needs. As User 100 uses the device Controller 150 will determine if it is appropriate to dose User 100 with the Beneficial Substance 105, based on the Beneficial Substance Use Regimen 115. And if it is Controller will dose User 100 using the Beneficial Substance Dosing Means. Software 140 running on Controller 150 will review Beneficial Substance Use Regimen 115 against User 100 actual use data which can be stored in Memory 155 and/or Data Storage 145 and will determine if User 100 is in compliance with Beneficial Substance Use Regimen 115, or not in compliance with Beneficial Substance Use Regimen 115. A user can be under or over compliant with the Beneficial Substance Use Regimen 115. Under-compliant means the user is taking less than the required doses of Beneficial Substance 105 based on the Beneficial Substance Use Regiment. While over-compliant means the user is taking too much of the Beneficial Substance 105 based on the Beneficial Substance Use Regimen 115. If User 100 is under-compliant then Controller will dose Addictive Substance 120 along with the Beneficial Substance 105. The effect is User 100 desire to use the device will increase.

If User 100 is over-compliant then Controller will dose Beneficial Substance 105 without Addictive Substance 120.

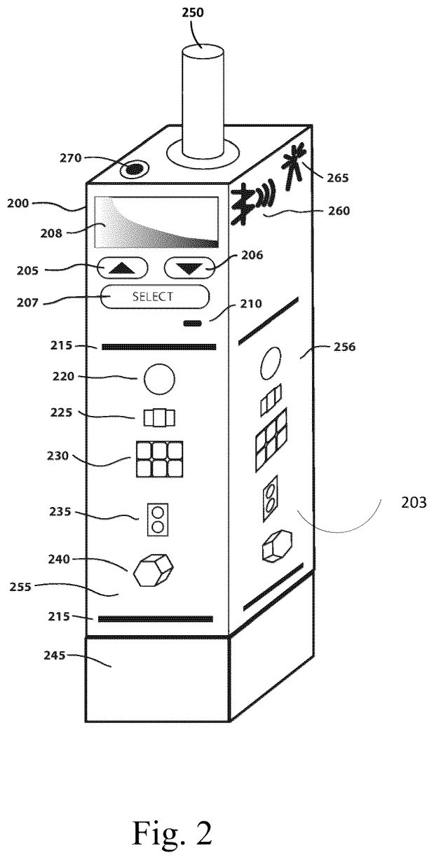

Referring to FIG. 2, an exemplary device is shown in a perspective view of the device housing and external components. Atomizer/Vaporizer Device has Controls including buttons to Select, Scroll Next, Scroll Previous, and a Display to see menus, menu options, and device parameters. Atomizer/Vaporizer Device has a Speaker to alert and notify the User. A cartridge (not shown) attaches to Atomizer/Vaporizer Device by connecting to the connector which aligns Substance IN-PORT, LED, Data Port, Optical Encoder, and Drive Shaft. A connected Battery provides power.

When User presses the Power Button the device powers up. A controller (not shown) checks for cartridges by reading the data port. If a cartridge (not shown) is attached the data port will read the cartridge's associated data and determine if there is enough valid information to enable the cartridge to be used by the User. If the cartridge is valid the Atomizer/Vaporizer Device will notify the user which can be via a sound from the speaker, a visual indication from the LED, a message on the display, or a combination thereof.

A pressure sensor (not shown) in the device checks for User applied pressure at the mouthpiece OUT-PORT. And if pressure is sensed the device will determine the amount of cartridge substance (not shown) to dispense into the IN-PORT for atomization/vaporization and inhalation by the User via OUT-PORT.

The atomization/vaporization device calculates the correct amount of cartridge substance (not shown) using data from the cartridge's associated data which is read by the data port. Accurate calculations of dispensed substances can be made knowing the diameter of the cartridge feed tube and by reading the cartridge's flow data in coordination with the optical encoder that tells how far the drive shaft moved the cartridge's feed wheel (not shown).

The atomizer/vaporizer device captures use and other data that is stored and can be transmitted to other devices. Transfer means may include wired or wireless connections including, but not limited to, Bluetooth, WiFi, infrared, USB, or other common means of transmitting data.

The atomizer/vaporizer device is run by a controller (not shown) and can be run with 1 to many cartridges in multiple combinations containing different substances. The atomizer/vaporizer device dispenses cartridge substances according to the parameters included in the cartridge's data chip and can run checks to ensure the combination of cartridges and their associated substances pass a DUR (Drug Usability Review). This can be performed in real-time locally at the device or via the device interacting with a computer severe over the internet and the device's Wifi connection. It can also be connected to an app and parameters that control the device's operation can be modified, updated, changed, or confirmed.

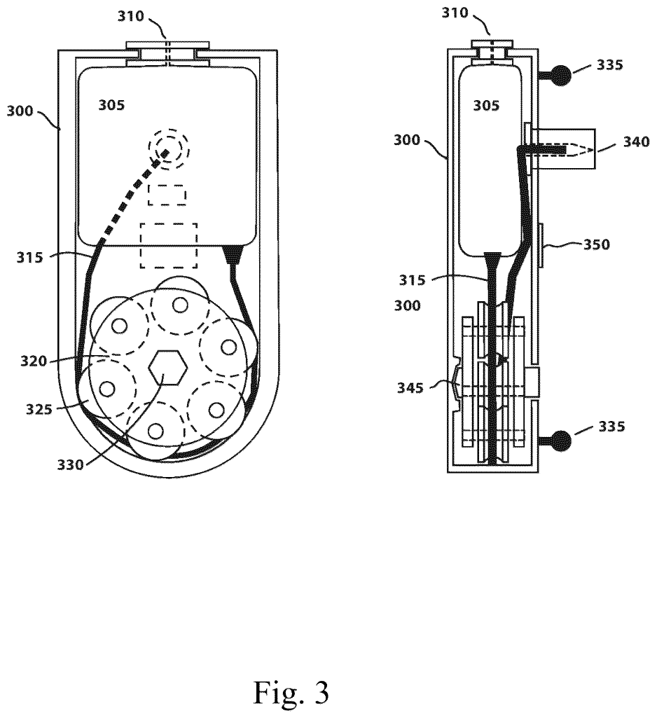

Referring to FIG. 3, an interior front and side view is shown of an exemplary cartridge. Cartridge contains a Substance Bladder with a fillable IN-PORT and a Rotor with attached Rollers (or alternatively shoes) that interfaces with a Substance Feed Line for the purposes of the on-command delivery of the Substance to the aerosolization mechanism inside the atomizer/vaporizer device (show in FIG. 2). The cartridge is attached to the atomizer/vaporizer device by snap connects that enable it to be attached and detached as needed. The snap connectors also align and supply the pressure needed for the data port to read and write to the cartridge data chip.

When attached a cartridge to the atomization/vaporization device a controller (not shown) reads the cartridge data chip and determines how, when, and if it can use the cartridge. The controller notifies the user if there is a problem with the cartridge or the substance, eg cartridge is expired, the cartridge is empty, the substance in the cartridge is not compliant with the other cartridges, the substance is unable to be properly dosed in the current device, and other such reasons for the cartridge to be unauthorized for use. Otherwise, the atomizer/vaporizer will notify the user that the cartridge is authorized for use.

When the atomization/vaporization device determines the cartridge is authorized for use it may use the cartridge in the following manner. When the atomization/vaporization device sense the pressure of a user inhaling from the atomization/vaporization device's OUT-PORT the controller will drive the Roller the rotations required to dispense the correct dose using the optical markings on the Rotor and the optical encoder in the atomizer/vaporizer device. The dose of the substance will be ejected out of the Cartridge OUT-PORT. Correct dosing is determined by data in the Cartridge Data Chip that is attached to the cartridge and updated when the bladder is filled.

The LED Lens is used to indicate different modes of operation and can also be used for decoration. For example, if there is a cartridge error it will be beneficial to the user to know which cartridge the error relates to. Flashing the cartridge associated with the error the color red will instantly help the user find the right cartridge. Lighting a cartridge green could tell the cartridge is ready for use. And other colors can also be tied to meaningful designations like yellow meaning “almost out, time to reorder”, blue meaning “take with water”, and other conditions that can be programmed into the atomization/vaporization device and/or the cartridge data chip and then used by the atomization/vaporization device to communicate valuable information about the cartridge to the user.

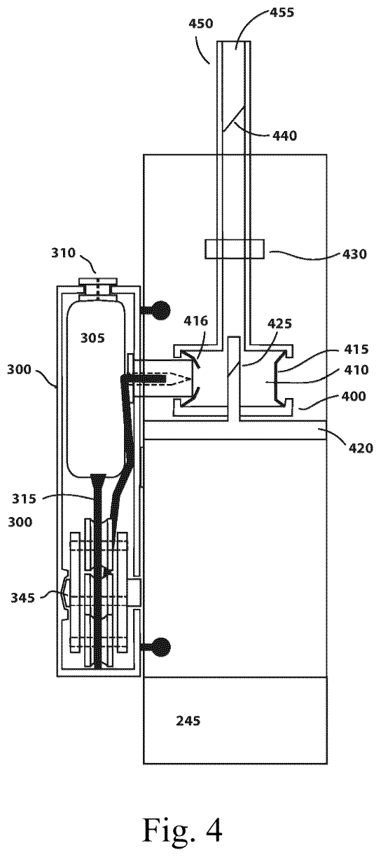

Referring to FIG. 4, a front interior view of an exemplary device with a cartridge attached is shown. When the User and device are ready to use the user inhales on the Mouthpiece which is sensed by the pressure sensor. The controller heats up Heating Element and Dispenses Substance from the Cartridge, shown on one side, into the Heating Chamber. The Heating Element vaporizes the substance and mixes it with air from the Air Intake Aperture. The vapor opens the check valves and is inhaled by the user.

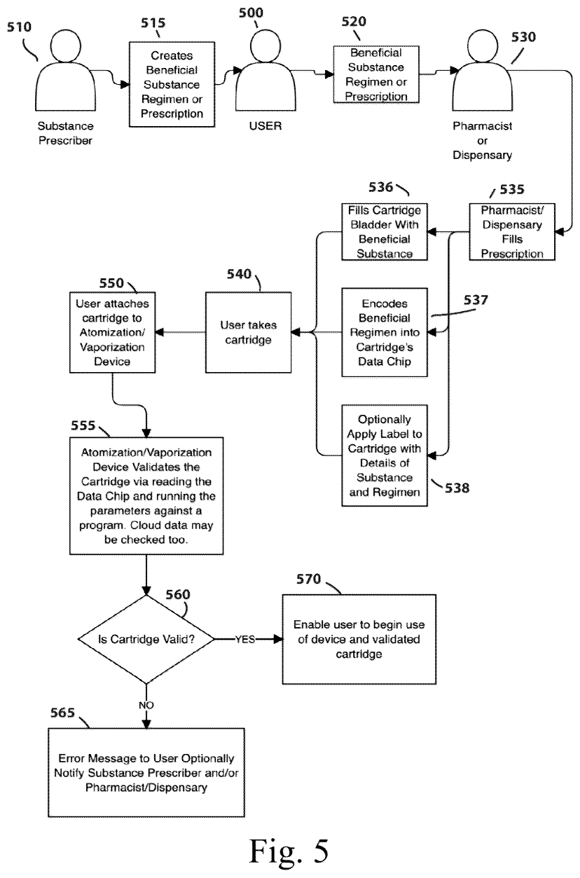

Referring to FIG. 5, an exemplary system flow chart is shown. The exemplary steps are as represented in FIG. 5.

Referring now to FIG. 6, an exemplary method steps schematic is shown. The method begins at start 572. Selection of a therapeutic regimen 574 sets the beneficial substances, doses, and frequencies. This information is used to provision the dosing device 576. After the dosing device is provisioned and given 578 to the user 580. The user begins using the dosing device 600.

The dosing device will begin dosing a dependency agent, such as nicotine or some other addictive agent, to create a dependency on the user to use the device. While the device is capable of dosing both the dependency agent and the beneficial substance each time the user uses the device. The device will freely dose the dependency agent but not the beneficial substance.

The device instead, manages the user adherence to the therapeutic regimen by only dosing the beneficial substance in accordance with the therapeutic regimen. This means the user can use the device to satisfy the cravings created by the dependency agent without being dosed their beneficial substance unless the dose complies with the beneficial regimen.

For example, consider a low-dose aspirin beneficial regimen. When the user wakes up to a new day and uses their dosing device for the first time that day, the device will dose the user both the addictive substance and an 81 mg dose of aspirin. The user is now adherent to their beneficial regimen for the day. Additional uses of the dosing device are allowed because the user will only receive doses of their dependency agent, The user cannot overdose or underdose their beneficial substance because the dosing device will only dose the beneficial substance per the therapeutic regimen.

Regimen Adherence Administration 601 will not dose an additional dose of aspirin until the therapeutic regime says to. Which is, in this example, the next day.

At some point in time there may be a reason to stop using the dosing device. For example, with some antibiotics, the therapeutic regimen's frequency might be taking two doses on day one, and then one dose each day for two weeks. In this example the dosing device would dose the beneficial substance twice on day one, and then once a day for two weeks. At the end of the two weeks the decision diamond 604 would determine that the regimen is no longer necessary and it would end. Thus, it would no longer dose any of the beneficial substance. And depending on the needs of the system the dosing device either continue to dose the addictive agent or not.

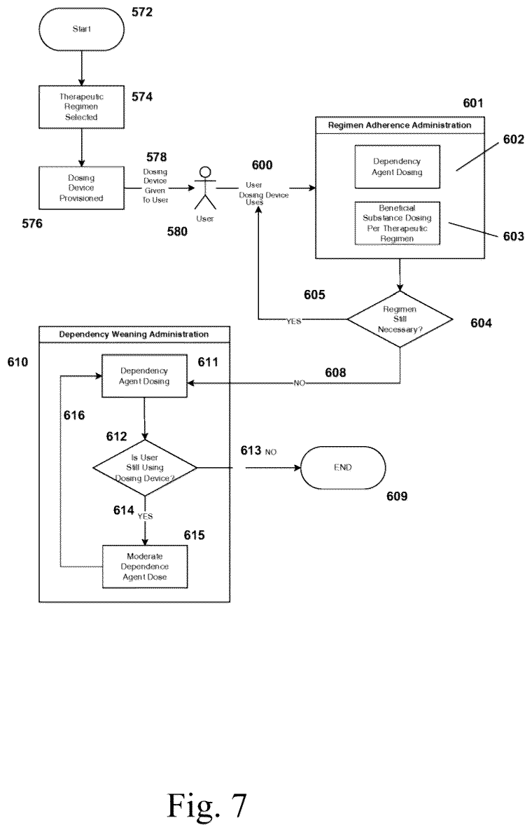

Referring now to FIG. 7, an exemplary method steps schematic with an additional weaning step is shown. In this system, after a therapeutic regimen is no longer needed, the method goes into a weaning mode. Dependency agent dosing 611 continues. A new decision diamond now checks to see if the user is still using the device 612. If the user is not using the device, then the system exits 613 and ends 607. If however, the user is still using the device 614 then the method moderates dependent agent dose 615 and via 616 loops back to allow dependency agent dosing 611.

Moderation of agent dose will depend on the implementation of the system and can be performed in various ways. For example, moderation of dependent agent dose could be the continuous lowering of the dose of the dependency agent which would effectively wean the user off the dosing agent. Moderation could also be performed by skipping doses. Or a combination of lowering and skipping of the doses of the dependency agent.

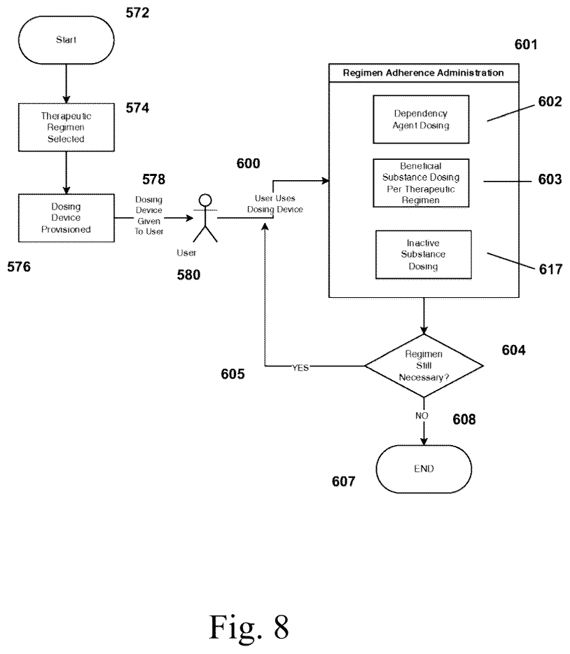

Referring now to FIG. 8, alternative exemplary method steps are shown. This enables the dosing device program to moderate the device dependency in addition to maintaining adherence to the therapeutic regimen. The user can now be dosed dependency agents, beneficial substances, inactive substances, or any combination of thereof.

Adding inactive substance dosing to regimen adherence administration enables the dosing device to moderate the addiction more precisely. If the user is using the dosing device in excess of what is needed to be adherent to the therapeutic regimen the dosing device could dose the inactive substance instead of the dependency agent. This will allow the user to use the device while also moderating the dependency.

If the user is using the dosing device too little the dosing device will increase the dose of the dependency agent. Conversely if the user is using the device too much then the dosing device needs to lower the doses of the dependency agent.

However, this may be impossible if the only options for using the dosing device are dosing the beneficial substance, the dependency agent, or a combination thereof.

While the device can moderate the dosing of the dependency agent, without another options, is still dosing the dependency agent. And the user could circumvent the lower dosage by increasing the number of times they use the dosing device.

To avoid this problem, we add an inactive substance. This enables the dosing device to moderate the doses of the dependency agent too. Now the dosing device and enable the user to use it without needing to give any of the dependency agent at all.

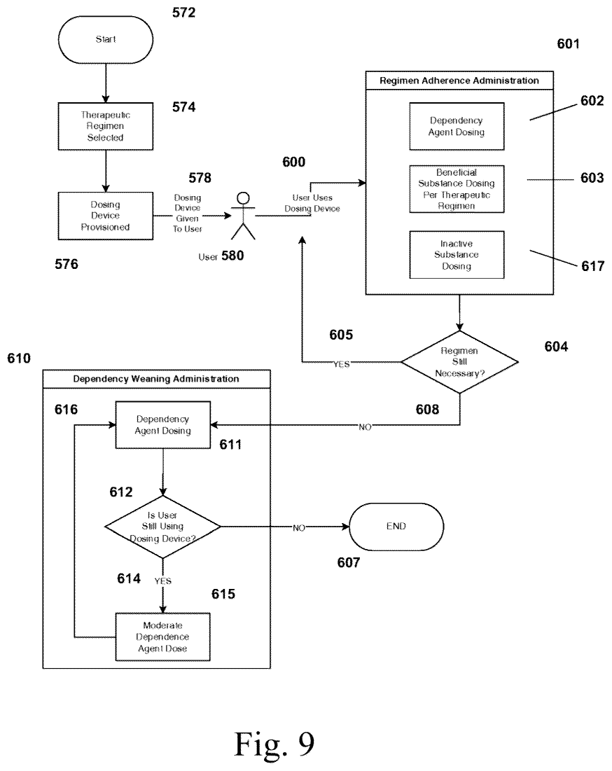

Referring now to FIG. 9, a schematic including an inactive ingredient and a weaning step is shown. The inactive substance will be exceptionally beneficial during dependency weaning administration. The dosing device can optimize the weaning process by supplementing the user's doses so they are the same quantity of substance but instead of being 100% dependency agent they can be a mixed percentage with part being the inactive substance. The experience of using the dosing device will feel the same if the user is, for example, receiving 90% dependency agent and 10% inactive substance. Or even 100% inactive substance and 0% dependency agent.

The physiological cravings would be felt if the dosing device simply replaced the dependency agent with the inactive substance. But by moderating the dependency agent and supplementing the lower dependency agent doses with the inactive substance the user will feel the dose is the same and the cravings will be satisfied as the doses are lowered, over time, till the user is weaned of the dependency agent.

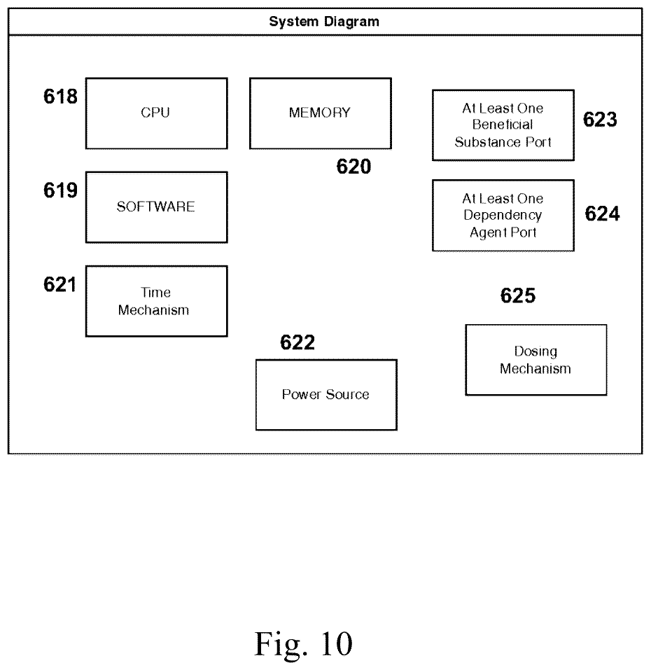

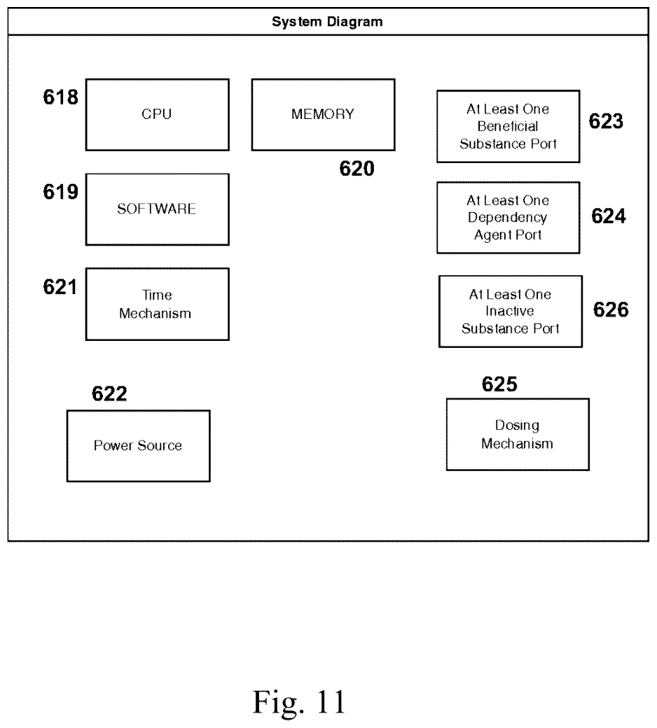

Referring now to FIGS. 10 and 11, a schematic with a delay timer function is shown. The figure shows a system diagram including CPU 618, software 619, memory 620, time mechanism 621, power source 622, beneficial substance port 622, dependency agent port 624, and a dosing mechanism 625. The therapeutic regimen is stored in memory 620, and the CPU 618, runs the software 619 that administers the dosing of beneficial substances according to the therapeutic regimen via the dosing mechanism 625.

The dosing mechanism 625 could be a heat vaporizer, ultrasonic vaporizer, a pump, valve, thermal bubble, drops, piezoelectric, or other known mechanisms to dose vapor, fumes, aerosols, liquids, gels, powders, pills, and beneficial substances in other forms. The time mechanism 621 could be an RC circuit, software-based timer, CPU-based timer, real time clock module, a software routine querying a time source through a website, API, satellite, other any other valid time source. Time is used to ensure the dose frequency is performed in accordance with the therapeutic regimen. The power source 520 works to operate the dosing device and all its components.

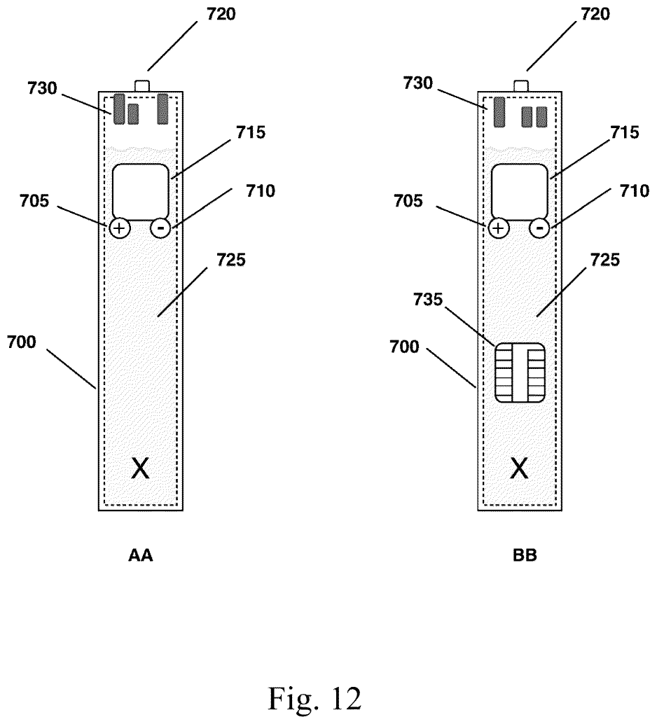

Referring now to FIG. 12, exemplary substance cartridges are shown. Cartridge AA includes case 700, positive vaporizer element contact 705, negative vaporizer element contact 710, vaporizer element 715, vapor out port 720, substance vessel 725, and key 730. The BB version includes everything AA does plus a data storage device 735.

These cartridges contain the beneficial substances, dependency agents, and inactive substances. They are filled when the dosing device is provisioned for the user. Provisioning includes filling the substance cartridges with the correct substances, installing them into the dosing device, and configuring the dosing device with the therapeutic regimen such that the dosing device's controller knows how to correctly apply the therapeutic regimen to the precise cartridges intended.

The dosing device, in order to keep the user adherent to their therapeutic regimen must match the correct cartridge with the correct therapeutic regimen. This can be done in various ways. One example attaches data storage devices, like a smart cards with information specific to the cartridge, to the cartridge. The dosing device's software reads the cartridge and applies the correct therapeutic regimen to each cartridge based on the data read from the cartridge.

Another example is to use keyed 730 cartridge cases, as shown in FIG. 7 AA and BB, like some inkjet printers do with ink cartridges to avoid placing the wrong ink in the wrong slot. This method ensures the dosing device knows which slot contains which substance and then applies the therapeutic regimen based on the slot of the dosing device.

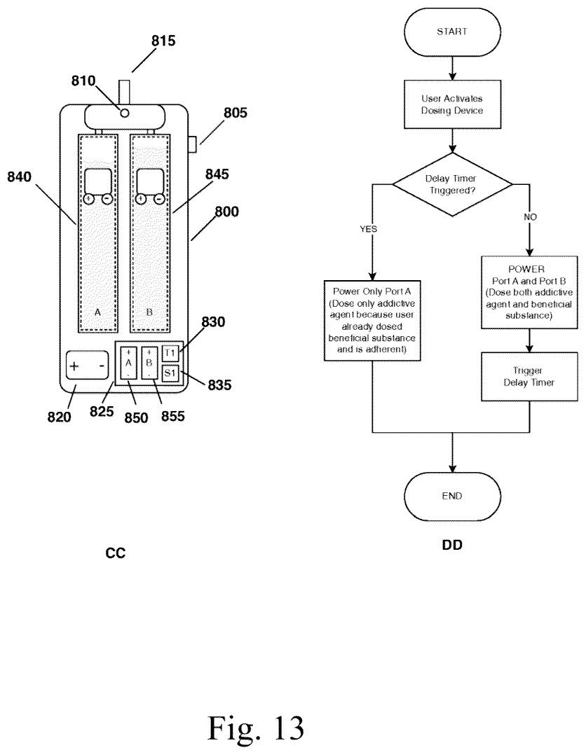

Referring now to FIG. 13, an exemplary device is shown. This embodiment of dosing device is a vape. The therapeutic regimen requires the user takes one dose of beneficial substance daily. The device was provisioned with a timer element 830 that is configured to, when triggered, remain triggered for 24 hours. Using a simple transistor the delay timer can control if the beneficial substance will be dosed.

The user can activate the device by pressing the activation button 805. Or the user can simply inhale/suck on the mouthpiece 815 like it's a straw. The sensor 810 can also activate the device based on the inhaling on the mouthpiece. The activation button 805 is not necessary to use the device but is shown as an option for devices that have indicators the user may wish to see without wishing to take a dose from the dosing device.

When the user inhales on the mouthpiece 815, the sensor 810 changes state and that state change sends power from the power source 820 to the controller 825. If the delay timer is NOT triggered the power will go to both Port A 850 and Port B 855, and the user will be dosed with both the addictive agent and the beneficial substance. And the timer element will trigger a 24 hour delay timer. The sensor 815 will revert back to its waiting state.

When the user next uses the dosing device, sensor 815 will again change state and send power to the controller 825. If the user is using the device while the timer element is triggered, then the controller 825 will block power from reaching port B 855 but not port A 850. Thus the user will be dosed with the addictive agent and not with the beneficial substance. No matter how often the use uses the dosing device the device will keep the user adherent to their therapeutic regimen.

The timing element can also adjust dosage by adjusting the length of time that power is sent to Port A 850 or Port B 855. This time based dose adjustment is determined during provisioning and based on the required dose per the therapeutic regimen. For clarity, it is not shown in the flowchart DD.

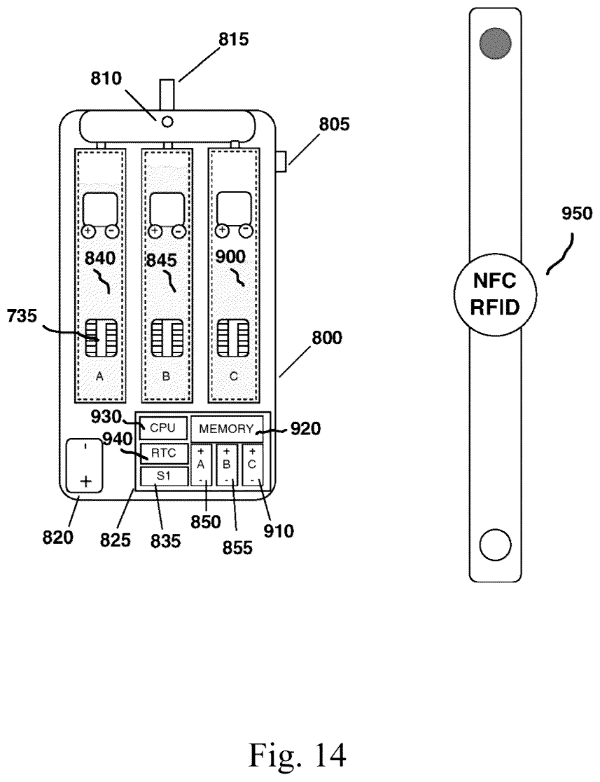

Referring now to FIG. 14, an exemplary device with security fob is shown. The figure shows a dosing device with a controller 825, that uses a CPU 930, real-time clock 940, memory 920, and an inactive substance cartridge with smart card data capabilities 900 in addition to the mouthpiece 815, sensor 810, activation button 805, addictive agent cartridge with smart card data capabilities 840, beneficial substance cartridge with smart card data capabilities 845, case 800, power source 820, sensor port 835, beneficial substance port B 855, addictive agent port A 850, inactive substance port C 910, and a security fob bracelet 950.

The dosing device of FIG. 9 is better able to manage the user experience because in addition to ensuring the user is adherent to their therapeutic regimen the CPU can modulate the dosing of all three cartridges. The goal of adherence with the best outcome for the user can be optimized by regulating the dosing of the addicting agent to be strong enough to enforce the user using the device in such a manner that the user has ample opportunity to take doses of their beneficial substance but not so much that the dependency on the device takes a toll on the user. If the user is using the device to excess, more than needed to dose the beneficial substance consistently and adherently, then the dosing device can modulate the dose of the addicting agent to lessen the dependency. Using the inactive substance means the user will experience a full dose and may not even know that the dependency agent does was lowered or that it may have been fully replaced by the inactive substance.

The ability to use the inactive substance as a stand in for dosing the dependency agent, in whole or in part, gives this dosing device an ability to optimize the dependency while therapy is necessary and makes it easier to wean the user off the dependency when the therapeutic regime is no longer necessary.

This version uses data storage enabled735 cartridges. This is similar to who printer companies use smart chip on their ink cartridges. By adding relevant data storage devices to the substance cartridges, the dosing device can ensure the authenticity of the cartridges as well as that they match the requirements of the provisioned therapeutic regimen.

Because this version has a CPU 930 it is easy to add a secure fob reading device to limit device use to only the person with the correct fob. In FIG. 9 we show a security fob bracelet 950 that would unlock the device when near. And would lock the dosing device when undetected.

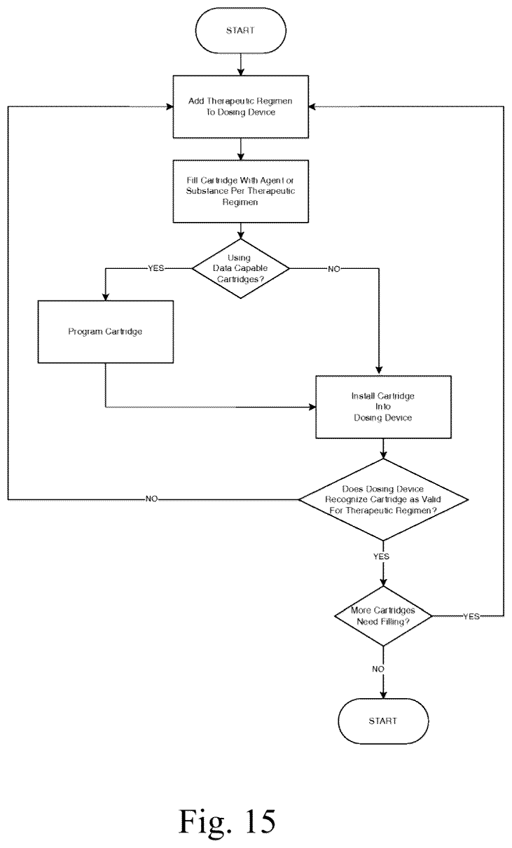

Referring now to FIG. 15, exemplary method steps for a provisioning procedure are shown. This version of the process begins with adding the therapeutic regimen to the dosing device. This gives the advantage that upon installation of the substance cartridges into the dosing device we can confirm that the dosing device is able to read the substance cartridges and that there are no errors.

A number of embodiments of the present disclosure have been described. While this specification contains many specific implementation details, these should not be construed as limitations on the scope of any disclosures or of what may be claimed but rather as descriptions of features specific to particular embodiments of the present disclosure.

Certain features that are described in this specification in the context of separate embodiments can also be implemented in combination in a single embodiment. Conversely, various features that are described in the context of a single embodiment can also be implemented in combination in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while methods and steps are depicted in the drawings in a particular order, this should not be understood as requiring that such operations be performed in the particular order shown or in sequential order, or that all illustrated operations be performed, to achieve desirable results.

Thus, particular embodiments of the subject matter have been described. Other embodiments are within the scope of the following claims. In some cases, the actions recited in the claims can be performed in a different order and still achieve desirable results. Nevertheless, it will be understood that various modifications may be made without departing from the spirit and scope of the claimed disclosure.

What is claimed is:

1. A method of promoting adherence to a therapeutic regimen, the method comprising:
creating a dependency-inducing motivation for a user to operate a dosing device;
detecting an instance of user operation of the dosing device; and
automatically administering, during the instance of user operation, a dose of a beneficial substance in accordance with a therapeutic regimen.

2. The method of claim 1, further comprising: providing the dosing device configured to deliver an addictive substance and a beneficial substance, and wherein creating the dependency-inducing motivation comprises establishing a dependency on the addictive substance sufficient to bias the user to operate the dosing device.

3. The method of claim 1, further comprising: receiving therapeutic regimen data defining a dose and timing for the beneficial substance, monitoring user interaction with the dosing device, and releasing the beneficial substance according to the therapeutic regimen while releasing an addictive substance according to an addictive-substance regimen.

4. The method of claim 1, further comprising: reducing an amount of the addictive substance delivered over time while maintaining delivery of the beneficial substance to transition the user from dependency-based adherence to non-dependency-based adherence.
